(19) United States
(12) Patent Application Publication
(10) Pub. No.: US 2023/0277868 A1
(43) Pub. Date: Sep. 7, 2023

(54) PHOTOTHERAPY METHOD, PHOTOTHERAPY DEVICE, AND PHOTOTHERAPY SYSTEM

(71) Applicant: OLYMPUS CORPORATION, Tokyo (JP)

(72) Inventor: Susumu YAMASHITA, Tokyo (JP)

(73) Assignee: OLYMPUS CORPORATION, Tokyo (JP)

(21) Appl. No.: 18/115,088

(22) Filed: Feb. 28, 2023

Related U.S. Application Data
(60) Provisional application No. 63/315,174, filed on Mar. 1, 2022.

(51) Int. Cl. A61N 5/06

(52) U.S. Cl. CPC A61N 5/062; A61N 5/0616; A61N 2005/0628; A61N 2005/0662

(57) ABSTRACT

A phototherapy method comprising: irradiating an affected area that is administered with a phototherapeutic agent with therapeutic light in a first period; and irradiating the affected area with the therapeutic light in a second period after the first period, wherein the second period is a period after the intensity of a first light that is emitted from the phototherapeutic agent has significantly decreased by irradiation with the therapeutic light in the first period, and an irradiation energy dose of the therapeutic light in the second period is determined based on a state of the affected area.

### CROSS REFERENCE TO RELATED APPLICATION

This application claims the benefit of U.S. Provisional Application No. 63/315,174, filed Mar. 1, 2022, which is hereby incorporated by reference herein in its entirety.

### TECHNICAL FIELD

The present disclosure relates to a phototherapy method, a phototherapy device, and a phototherapy system.

### BACKGROUND

Photoimmunotherapy (PIT) is a technique for treating cancer with photosensitive agents combined with therapeutic light irradiation. Conventionally, phototherapy is started with use of high-power therapeutic light, and the timing to end the phototherapy is determined based on the irradiation time determined for each affected area.

In the PIT, it is important to manage an irradiation energy dose of therapeutic light since excessive irradiation with the therapeutic light may cause side effects, while insufficient irradiation leads to insufficient therapy. Accordingly, techniques for evaluating a progress state of phototherapy based on the intensity of fluorescence emitted from an affected area have been proposed (see, for example, Patent Literature 1). Patent Literature 1 discloses prevention of excessive irradiation with therapeutic light, by detecting fluorescence, generated by irradiation with therapeutic light, from an affected area, and automatically terminating the irradiation with therapeutic light or displaying a termination message on a display unit, when the intensity of the fluorescence reaches a prescribed value or less.

### SUMMARY

One aspect of the present disclosure is a phototherapy method comprising: irradiating an affected area that is administered with a phototherapeutic agent with therapeutic light in a first period; and irradiating the affected area with the therapeutic light in a second period after the first period, wherein the second period is a period after the intensity of a first light that is emitted from the phototherapeutic agent has significantly decreased by irradiation with the therapeutic light in the first period, and an irradiation energy dose of the therapeutic light in the second period is determined based on a state of the affected area.

Another aspect of the present disclosure is the first period is a period where the intensity of the first light significantly decreases after irradiation with the therapeutic light is started.

Another aspect of the present disclosure is the second period is a period including a state where the intensity of the first light is substantially constant.

Another aspect of the present disclosure is a phototherapy device comprising: an irradiation unit light source configured to irradiate an affected area that is administered with a phototherapeutic agent with therapeutic light; and a processor comprising hardware, the processor being configured to: measure light intensity of the affected area that is irradiated with the therapeutic light; control irradiation of the affected area with the therapeutic light, based on the measured light intensity of the affected area; and additionally irradiating the affected area with the therapeutic light of a specified irradiation energy dose, after the light intensity of the affected area has significantly decreased.

Another aspect of the present disclosure is a control apparatus comprising: a processor comprising hardware, the processor being configured to: control irradiation of an affected area with a therapeutic light, based on a measured fluorescence intensity of the affected area, and control an irradiation light source to additionally irradiate the affected area with the therapeutic light of a specified irradiation energy dose, after: the fluorescence intensity decreases below a predetermined threshold or an amount of change in the fluorescence intensity per unit time is less than the predetermined threshold.

### BRIEF DESCRIPTION OF DRAWINGS

FIG. 1 is an overall configuration diagram of a phototherapy system according to an embodiment of the present disclosure.

FIG. 2 is a graph showing time change in fluorescence intensity of a phototherapeutic agent due to irradiation with therapeutic light, in which the relationship between a total irradiation energy dose (horizontal axis) of the therapeutic light and the fluorescence intensity (vertical axis) of the phototherapeutic agent is expressed.

FIG. 3 is a flowchart showing a phototherapy method according to the embodiment of the present disclosure.

FIG. 4A is a graph showing the relationship between the total irradiation energy dose (horizontal axis) of the therapeutic light and the fluorescence intensity (vertical axis) of the phototherapeutic agent acquired in an experiment.

FIG. 4B is a graph showing the relationship between the total irradiation energy dose (horizontal axis) of the therapeutic light and the fluorescence intensity (vertical axis) of the phototherapeutic agent acquired in an experiment.

FIG. 4C is a graph showing the relationship between the total irradiation energy dose (horizontal axis) of the therapeutic light and the fluorescence intensity (vertical axis) of the phototherapeutic agent acquired in an experiment.

FIG. 5 is a table showing the result of pathologically evaluating the phototherapeutic effect on tumors in the experiments in FIGS. 4A to 4C, in which the relationship between the total irradiation energy dose of the therapeutic light and the phototherapeutic effect is expressed.

FIG. 6 is a flowchart showing the phototherapy method according to another embodiment of the present disclosure.

### DETAILED DESCRIPTION

A phototherapy method, a phototherapy device, and a phototherapy system according to an embodiment of the present disclosure will be described with reference to the drawings.

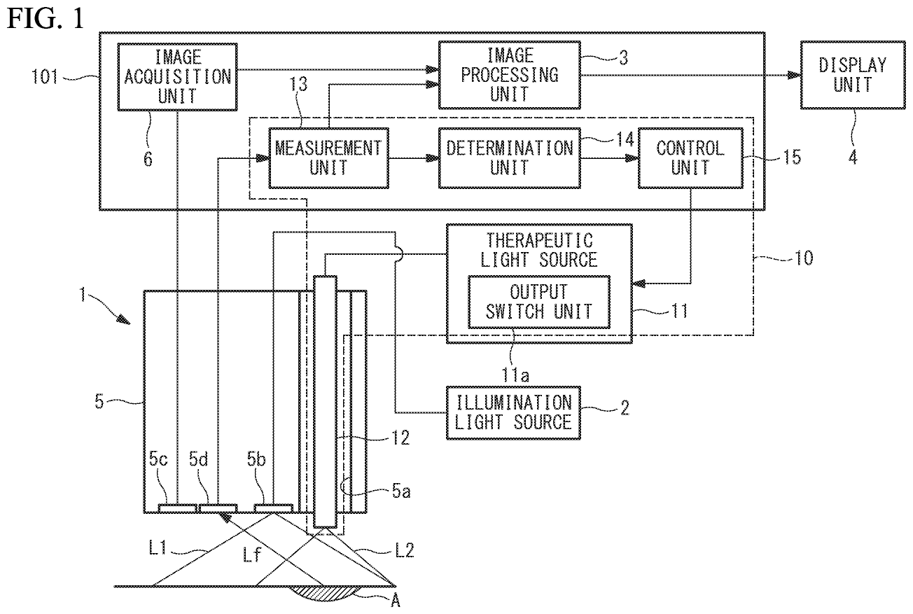

As shown in FIG. 1, a phototherapy system 100 according to the present embodiment is an endoscope system that optically treats an affected area A using a light-responsive phototherapeutic agent, while observing the affected area A with an endoscope 1. The affected area A is, for example, a tumor such as cancer. The phototherapeutic agent, which is made of fluorescent molecules characterized in accumulating in the affected area A, demonstrates phototherapeutic effects through photochemical reactions induced by excitation light. The phototherapeutic agent is, for example, a panitumumab-IR700 complex or a hematoporphyrin derivative.

The phototherapy system 100 includes the endoscope 1 for observing the affected area A inside a patient's body, an illumination light source 2 that generates white illumination light L1 to illuminate the affected area A, an image processing unit (image processor) 3 that processes an endoscopic image, a display unit 4 (display) that displays the endoscopic image, and a phototherapy device 11. The endoscope 1 has a proximal end connected to an endoscope processor 101.

The endoscope 1 includes a flexible or rigid elongated scope 5 and an image acquisition unit 6 having an image sensor. The scope 5 includes a treatment instrument channel 5a extending through the scope 5 in a longitudinal direction thereof. The scope 5 has a front end face provided with an illumination window 5b and a light receiving window 5c. The endoscope 1 emits illumination light L1 supplied from the illumination light source 2 to the affected area A through the illumination window 5b. The endoscope 1 receives, at the light receiving window 5c, the illumination light L1 reflected on the affected area A, and acquires an endoscopic image of the affected area A by the image acquisition unit 6.

The image processing unit 3 receives the endoscopic image from the image acquisition unit 6, processes the endoscopic image as necessary, and outputs the endoscopic image to the display unit 4.

The display unit 4 is a display device including any kind of display, such as a liquid crystal display.

The phototherapy device 11 includes a therapeutic light source 11 that generates therapeutic light L2 to treat the affected area A, a probe (irradiation unit) 12 that irradiates the affected area A with the therapeutic light L2 inserted into the body via the treatment instrument channel 5a, a measurement unit 13 that measures fluorescence intensity F of the affected area A that is irradiated with the therapeutic light L2, a determination unit 14 that determines a first period P1 and a second period P2 based on the measured fluorescence intensity F, and a control unit 15 (controller) that controls the therapeutic light source 11 based on the result of determination by the determination unit 14.

The phototherapy device 10 includes a processor such as a CPU, a memory such as a RAM, and a computer-readable non-transitory recording medium such as a ROM or a HDD. The processor, the memory and the recording medium are provided, for example, inside the endoscope processor 101. The recording medium stores a phototherapy program for causing the processor to execute a phototherapy method described later. The processor implements later-described processing of the determination unit 14 and the control unit 15.

The therapeutic light source 11 outputs the therapeutic light L2 that is excitation light having an excitation wavelength of the phototherapeutic agent. The therapeutic light source 11 may have an output switching unit 11a that changes output power of the therapeutic light L2, so that the output power of the therapeutic light L2 may be changeable according to the control of the control unit 15.

A probe 12, which is an elongated optical fiber probe having an optical fiber to guide the therapeutic light L2, is inserted into the treatment instrument channel 5a. The probe 12 has a proximal end connected to the therapeutic light source 11, and the therapeutic light L2 is emitted from a distal end of the probe 12 to irradiate the affected area A.

A plurality of probes 12 may be provided, and an amount of the therapeutic light L2 that irradiates the affected area A may be increased or decreased.

At the front end face of the scope 5, a barrier filter 5d is provided to selectively transmit fluorescence Lf while blocking the light that is different in wavelength range from the fluorescence Lf. The measurement unit 13 detects intensity F of the fluorescence Lf passing through the barrier filter 5d and transmits the fluorescence intensity F to the determination unit 14. For example, the measurement unit 13 has an image sensor provided in the endoscope processor 101 or in the scope 5 to acquire a fluorescent image including information on the fluorescence intensity F. The fluorescent image or the fluorescence intensity F may be displayed in real time on the display unit 4. The measurement unit 13 may have any kind of photodetector other than the image sensor.

The determination unit 14 determines the first period P1 and the second period P2 based on change of the fluorescence intensity F.

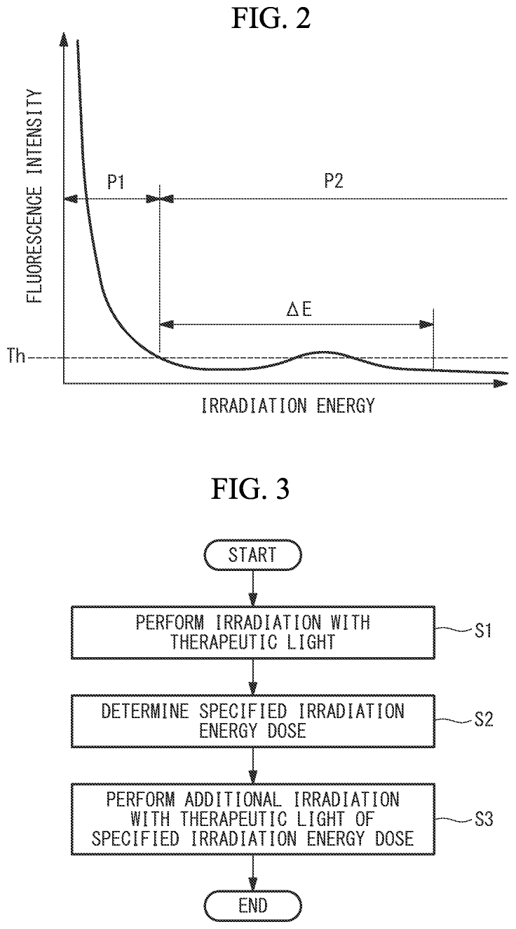

FIG. 2 shows time change in the fluorescence intensity F of the phototherapeutic agent due to irradiation with the therapeutic light L2. The process of the time change in the fluorescence intensity F includes the first period P1 at an early stage when the fluorescence intensity F significantly decreases, and the second period P after the first period P1. The second period P2 includes the state where the fluorescence intensity F is substantially constant and the state where the fluorescence intensity F gradually fluctuates.

The determination unit 14 determines the first period P1 and the second period P2 based on the magnitude of the fluorescence intensity F or a time change rate.

For example, the determination unit 14 determines the period from the start of irradiation with the therapeutic light L2 to the time when the fluorescence intensity F decreases to a prescribed threshold Th as the first period P1, and the period after the decrease of the fluorescence intensity F to the prescribed threshold Th as the second period P2. The result of determination by the determination unit 14 may be displayed on the display unit 4.

Alternatively, the determination unit 14 may determine that the period from the start of irradiation with the therapeutic light L2 to the time when the fluorescence intensity F decreases by a prescribed amount as the first period P1. For example, the fluorescence intensity F significantly decreasing in the first period P1 means the fluorescence intensity F decreases below the prescribed threshold Th or an amount of change in fluorescence intensity F per unit time becomes less than a predetermined threshold.

The control unit 15 controls the therapeutic light source 11 based on the result of a determination by the determination unit 14, so as to irradiate the affected area A with the therapeutic light L2 in the first period P1 and to irradiate the affected area A with the therapeutic light L2 of a specified irradiation energy dose ΔE in the second period P2. The specified irradiation energy dose ΔE is determined in accordance with the state of the affected area A. The specified irradiation energy dose ΔE is, for example, 1 J or more, and is set to an optimal value so that the total energy dose is 100 J/cm² or less in order to attain the phototherapeutic effect for the affected area A.

Now, a phototherapy method executed by the phototherapy system 100 is described.

As shown in FIG. 3, the phototherapy method includes step S1 of irradiating the affected area A that is administered with a phototherapeutic agent with the therapeutic light L2, step S2 of determining a specified irradiation energy dose ΔE, and step S3 of additionally irradiating the affected area A with the therapeutic light L2 of the specified irradiation energy dose ΔE.

In step S1, the control unit 15 controls so as to output the therapeutic light L2 from the therapeutic light source 11 to irradiate the affected area A with the therapeutic light L2.

In the affected area A that is irradiated with the therapeutic light L2, fluorescence Lf of the phototherapeutic agent is generated. The measurement unit 13 measures the fluorescence intensity F of the affected area A, and the determination unit 14 determines whether or not the phototherapy is in the first period P1 based on the change in the fluorescence intensity F. When, for example, the fluorescence intensity F falls to a prescribed threshold Th, the determination unit 14 determines that the first period P1 has ended.

In subsequent step S2, in response to the determination result of the determination unit 14, the control unit 15 determines the specified irradiation energy dose ΔE, and then starts step S3.

In step S3, the affected area A is additionally irradiated with the therapeutic light L2. At the time when irradiation with the therapeutic light L2 of the specified irradiation energy dose LE is performed after step S1, the control unit 15 controls the therapeutic light source 11 to stop irradiation of the affected area A with the therapeutic light L2, and the phototherapy of the affected area A with the therapeutic light L2 is ended.

Here, the relationship between the fluorescence intensity F and the phototherapeutic effect is described.

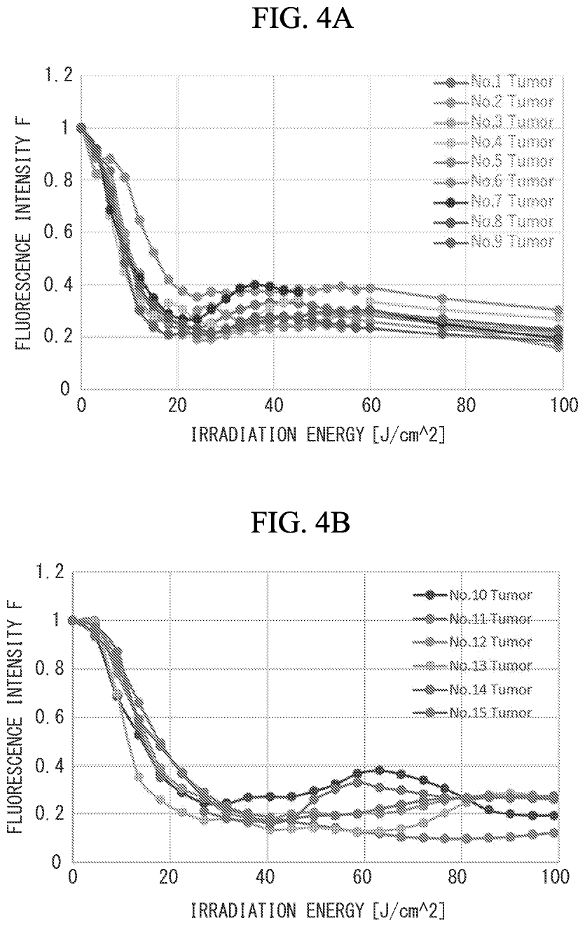

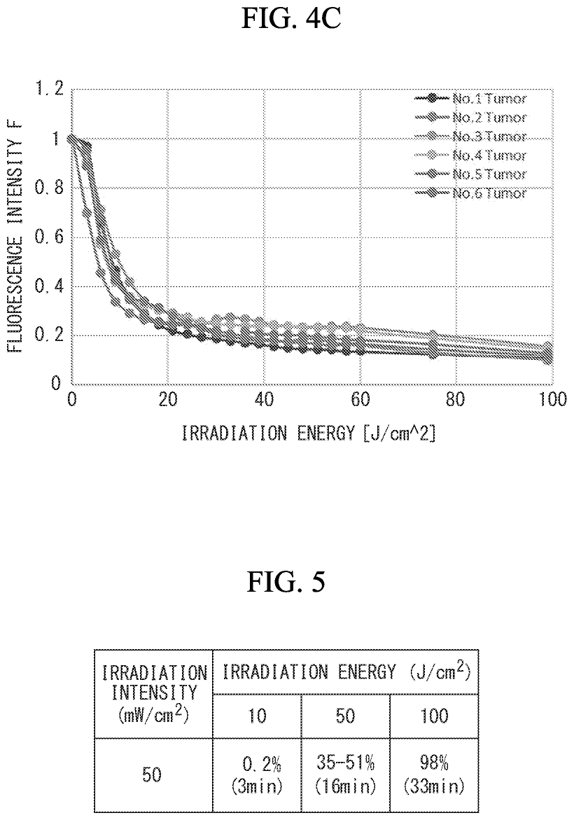

FIGS. 4A to 4C show the results of experiments to examine time change in the fluorescence intensity F of the phototherapeutic agent using tumor model animals. FIGS. 4A and 4B show experimental results in a skin cancer model, and FIG. 4C shows an experimental result in a gastric cancer model. In the experiments shown in FIGS. 4A, 4B, and 4C, the power (power density) of the therapeutic light L2 is 50 mW/cm², 150 mW/cm², and 50 mW/cm², respectively.

According to the experiments, it was found that the fluorescence intensity F of the phototherapeutic agent changes in two stages in accordance with the total irradiation energy dose of the therapeutic light L2. In FIGS. 4A to 4C, the horizontal axis represents the irradiation energy dose (J/cm²) of the therapeutic light L2, and the vertical axis represents the fluorescence intensity F.

Specifically, in the first period P1 where the total irradiation energy dose is 20 J/cm² or less, the fluorescence intensity F decreases significantly, and in the second period P2 where the total irradiation energy dose is more than 20 J/cm², the fluorescence intensity F is in a substantially constant state, and then fluctuates gradually. A constant state and a substantially constant state may include a state that the fluorescence intensity F remains below the prescribed threshold Th for a certain period of time or a state that fluorescence intensity F per unit time changes little.

Here, it can be determined that the fluorescence intensity F has decreased significantly when the fluorescence intensity F decreases by 60% or more from the fluorescence intensity F at the start of irradiation with the therapeutic light L2. Such a sharp decrease in the fluorescence intensity F means that the phototherapeutic agent is sufficiently working on the affected area A. On the other hand, when such a sharp decrease in the fluorescence intensity F does not appear, it is presumed that the phototherapeutic agent is not sufficiently working on the affected area A, and therefore, irradiation with the therapeutic light L2 is stopped, or the probe 12 is moved to the position of another affected area A.

Furthermore, when the same affected area A is continuously irradiated with the therapeutic light L2 in the first period P1, then the state of the fluorescence intensity changes to at least one of a constant state and a state having a similar inflection point. Based on this change, it can be determined that the phototherapy reaches the second period P2.

In this way, the time when the fluorescence intensity F changes from sharp attenuation state in the first period P1 to the state in the second period P2 is determined as the starting point of the second period P2. This makes it possible to irradiate the affected area with the therapeutic light L2 of the specified irradiation energy dose ΔE that ensures acquisition of the phototherapeutic effect.

FIG. 5 shows the result of pathologically evaluating the phototherapeutic effect on tumors in tumor model animals.

In the past, it was considered that phototherapy was in progress during the first period P1 where the fluorescence intensity F significantly attenuated. However, according to the result of evaluation in FIG. 5, in the first period P1 where the total irradiation energy dose of the therapeutic light L2 is small, there is less pathological changes one day after the therapy, so that a high phototherapeutic effect cannot be expected. Specifically, at the initial stage of irradiation with therapeutic light L2, a change amount of the fluorescence intensity F is large, though there is almost no phototherapeutic effect, and therefore the phototherapy is insufficient at the time when the fluorescence intensity F sufficiently decreases.

Furthermore, it was found that in the second period P2 after the fluorescence intensity F sufficiently decreases, the phototherapeutic effect increases with the increase of the total irradiation energy dose, though the fluorescence intensity F is in a substantially constant state or in the state of a gradual change.

Thus, an attenuation amount (attenuation rate) of the fluorescence intensity F does not uniquely represent the phototherapeutic effect. In addition, when the phototherapeutic agent is not sufficiently combined with the affected area A, the fluorescence intensity F may not decrease. Therefore, it is not possible to determine whether the phototherapeutic effect is attained based solely on the fluorescence intensity F.

According to the present embodiment, the irradiation process of the therapeutic light L2 is divided into two periods P1 and P2, and irradiation with the therapeutic light L2 is controlled in the first period P1 and the second period P2 based on criteria different from each other. Then, in the second period P2 after the fluorescence intensity F significantly decreases, the affected area A is additionally irradiated with the therapeutic light L2 of the specified irradiation energy dose ΔE. This ensures that the sufficient phototherapeutic effect can be acquired in the affected area A.

In the above embodiment, the power of the therapeutic light L2 may be different between the first period P1 and the second period P2. Appropriate power of the therapeutic light L2 may be different between the first period P1 and the second period P2. For example, a first power of the therapeutic light L2 in the first period P1 can be low enough that a change in the fluorescence intensity F is obtainable. On the other hand, a second power of the therapeutic light L2 in the second period P2 may be higher than the first power.

For example, a user may observe the fluorescence intensity F or the determination result regarding the periods P1 and P2 displayed on the display unit 4, and when a significant decrease in the fluorescence intensity F is recognized or the first period P1 shifts to the second period P2, the user may change the power of the therapeutic light L2. Changing the power of the therapeutic light L2 may be performed by switching the output power of the therapeutic light source 11 or by other means. For example, the power of the therapeutic light L2 that is irradiated on the affected area A may be changed by bringing the tip of the probe 12 closer to the affected area A. Here, a distance between the probe 12 and the affected area A may be obtained by checking the intensity of reflection light due to the illumination light L1 coming from the vicinity of the affected area A, on the display unit 4, or by checking with naked eye.

In the above embodiment, the control unit 15 may perform the following control in the first period P1 and second period P2.

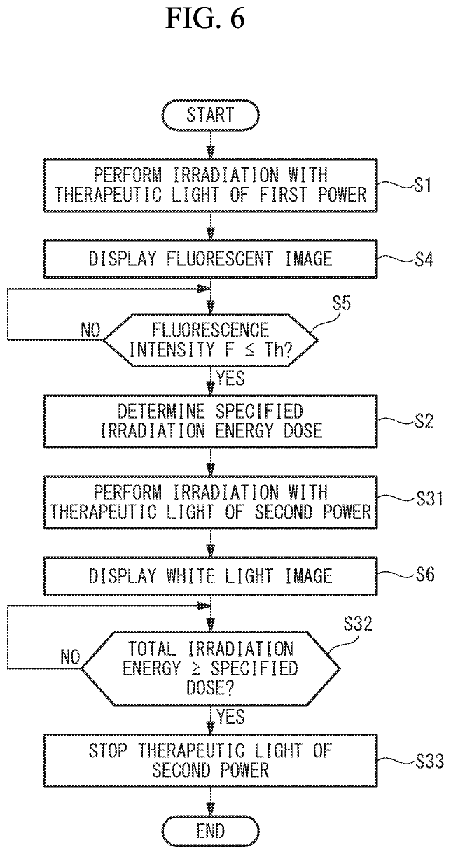

FIG. 6 shows another example of the phototherapy method executed by the phototherapy system 100. The phototherapy method further includes steps S4, S5 and S6 in addition to steps S1, S2, and S3.

After starting irradiation with the therapeutic light L2 of the first power (step S1), the control unit 15 displays information based on the fluorescence intensity F measured by the measurement unit 13 on the display unit 4 in real time (step S4). The information is, for example, a fluorescent image or the fluorescence intensity F. Therefore, during the first period P1, an operator can observe the information displayed on the display unit 4, and visually confirm the attenuation of the fluorescence F based on passage of irradiation time of the therapeutic light L2 or integrated irradiation energy.

During the first period P1, the determination unit 14 determines whether or not the fluorescence intensity F measured by the measurement unit 13 has decreased to a prescribed threshold Th (step S5). When determination is made that the fluorescence intensity F has decreased to the prescribed threshold Th (YES in step S5), the determination unit 14 determines that the first period P1 has ended, and the control unit 15 advances to step S2.

Step 3 subsequent to step S2 includes steps S31 to S33. The control unit 15 starts irradiation with the therapeutic light L2 of the second power (step S31). The second power may be identical to the first power and may be different from the first power.

After step S31, the control unit 15 controls the illumination light source 2 to output the white illumination light L1 from the illumination light source 2, and irradiate the affected area A with the white illumination light L1 in parallel with the irradiation with the therapeutic light L2 of the second power. Accordingly, the image acquisition unit 6 acquires an endoscopic image, which is a white light image of the affected area A, and the endoscopic image is displayed on the display unit 4 via the image processing unit 3 (step S6). Therefore, during the second period P2, the endoscopic image displayed on the display unit 4 allows the operator to observe the affected area A that is irradiated with the therapeutic light L2 under the white illumination light L1. In other words, during the second period P2, the operator can visually confirm the progress of the phototherapy of the affected area A using the therapeutic light L2 through the endoscopic image displayed on the display unit 4.

The control unit 15 measures the irradiation energy dose of the therapeutic light L2 from the start of irradiation with the therapeutic light L2 of the second power (step S32). When the total irradiation energy dose in the second period P2 reaches a specified dose (YES in step S32), the control unit 15 controls the therapeutic light source 11 to stop irradiation with the therapeutic light L2 (step S33). For example, when the specified dose is 100 J/cm², irradiation with the therapeutic light L2 is stopped once the total irradiation energy dose reaches 100 J/cm².

In step S33, the control unit 15 may perform operation of warning the operator that the total irradiation energy dose has reached the specified amount, instead of controlling the therapeutic light source 11 to stop irradiation with the therapeutic light L2. In this case, a warning is issued when the total irradiation energy dose reaches the specified dose (for example, 100 J/cm²). For example, the control unit 15 may display on the display unit 4 an indication indicating that the total irradiation energy dose has reached the specified dose. Alternatively, the control unit 15 may output sound, such as voice, to inform the operator that the total irradiation energy dose has reached the specified dose through a buzzer or a speaker, etc.

After confirming that the total irradiation energy dose has reached the specified dose based on the warning, the operator confirms the progress of the phototherapy with the endoscopic image that is acquired using the white illumination light L1 and displayed on the display unit 4. When the phototherapy is determined to be sufficient, the operator may perform an operation to stop the output of the illumination light L1 from the therapeutic light source 11.

The control unit 15 may stop irradiation with the therapeutic light L2, when the total irradiation energy dose reaches a prescribed percentage of the specified dose, that is, half the specified dose, for example. The operator may confirm the progress of phototherapy after the therapeutic light L2 is stopped, and may restart irradiation with the therapeutic light L2 when determining that the phototherapy is insufficient. Even in this case, the control unit 15 stops irradiation with the therapeutic light L2 when the total irradiation energy dose reaches the specified dose. In addition, after the total irradiation energy dose reaches the specified dose and irradiation with the therapeutic light L2 is stopped, the operator may confirm the progress of the phototherapy, and may perform additional irradiation with the therapeutic light L2 when determining that additional irradiation is necessary.

The control unit 15 may issue a warning before the total irradiation energy dose reaches the specified dose. For example, when the prescribed dose is 100 J/cm², the control unit 15 provides a visual or audible warning to the operator to confirm the progress of the phototherapy once the total irradiation energy dose reaches 20 J/cm². Then, when the total irradiation energy dose reaches 100 J/cm², the control unit 15 stops irradiation with the therapeutic light L2.

In this case, the control unit 15 may repeatedly provide the warning every time the total irradiation energy dose increments by a prescribed dose (for example, 20 J/cm²). When the operator does not attempt to stop the therapeutic light L2, the control unit 15 may continue irradiation with the therapeutic light L2, and stop irradiation with the therapeutic light L2 once the total irradiation energy dose has reached the specified dose.

### REFERENCE SIGNS LIST

4 Display unit
11 Phototherapy device
12 Probe (irradiation unit)
13 Measurement unit
14 Determination unit
15 Control unit
100 Phototherapy system
101 Endoscope Processor
A Affected area
L2 Therapeutic light
Lf Fluorescence
P1 First period
P2 Second period

What is claimed is:

1. A phototherapy method comprising:
irradiating an affected area that is administered with a phototherapeutic agent with therapeutic light in a first period; and
irradiating the affected area with the therapeutic light in a second period after the first period, wherein
the second period is a period after the intensity of a first light that is emitted from the phototherapeutic agent has significantly decreased by irradiation with the therapeutic light in the first period, and
an irradiation energy dose of the therapeutic light in the second period is determined based on a state of the affected area.

2. The phototherapy method according to claim 1, wherein the first period is a period where the intensity of the first light significantly decreases after irradiation with the therapeutic light is started.

3. The phototherapy method according to claim 2, wherein the second period is a period including a state where the intensity of the first light is substantially constant.

4. The phototherapy method according to claim 1, wherein power of the therapeutic light in the first period is low enough that a change in the intensity of the first light is obtainable.

5. The phototherapy method according to claim 1, further comprising:
observing information based on the intensity of the first light emitted from the affected area by irradiating the affected area with the therapeutic light in the first period;
irradiating the affected area with the therapeutic light and white illumination light, and observing the affected area that is irradiated with the therapeutic light under the white light in the second period; and
measuring the irradiation energy dose in the second period, and stopping irradiation with the therapeutic light in the second period when the irradiation energy dose reaches a predetermined dose.

6. A phototherapy device comprising:
an irradiation light source configured to irradiate an affected area that is administered with a phototherapeutic agent with therapeutic light; and
a processor comprising hardware, the processor being configured to:
measure light intensity of the affected area that is irradiated with the therapeutic light;
control irradiation of the affected area with the therapeutic light, based on the measured light intensity of the affected area; and
additionally irradiating the affected area with the therapeutic light of a specified irradiation energy dose, after the light intensity of the affected area has significantly decreased.

7. The phototherapy device according to claim 6, wherein
the irradiation light source is configured to perform irradiation with excitation light, and
the processor is configured to measure intensity of fluorescence emitted from the phototherapeutic agent as the light intensity of the affected area.

8. The phototherapy device according to claim 6, wherein the processor is further configured to determine a first period and a second period based on a change in the measured light intensity,
wherein the first period includes a state where the light intensity of the affected area significantly decreases, and the second period is a period after the first period.

9. The phototherapy device according to claim 6, wherein the processor is further configured to:
control a display to display in real time a change in the measured light intensity of the affected area, and
control the irradiation light source to additionally irradiate the affected area with the therapeutic light of a specified irradiation energy dose, after the light intensity of the affected area has significantly decreased.

10. The phototherapy system according to claim 9, wherein the processor is further configured to determine a first period and a second period based on the change in the measured light intensity,
wherein the first period includes a state where the light intensity of the affected area significantly decreases,
the second period is a period after the first period, and
the processor controls the display to display a result of the determination.

11. The phototherapy system according to claim 10, wherein
the processor is configured to
control the display to display information based on the light intensity of the affected area measured by the measurement unit in the first period,
control irradiation of the affected area with the therapeutic light and white illumination light and control the display to display an image of the affected area in the second period, and
measure the irradiation energy dose in the second period, and stop irradiation with the therapeutic light in the second period, when the irradiation energy dose reaches a predetermined dose.

12. The phototherapy system according to claim 10, wherein the processor is configured to
control the display to display information based on the measured light intensity of the affected area in the first period,
control irradiation of the affected area with the therapeutic light and white illumination light and control the display to display an image of the affected area in the second period, and
measure the irradiation energy dose in the second period, and issue a warning, when the irradiation energy dose reaches a predetermined amount.

13. A control apparatus comprising:
a processor comprising hardware, the processor being configured to: control irradiation of an affected area with a therapeutic light, based on a measured fluorescence intensity of the affected area, and
control an irradiation light source to additionally irradiate the affected area with the therapeutic light of a specified irradiation energy dose, after:
the fluorescence intensity decreases below a predetermined threshold or
an amount of change in the fluorescence intensity per unit time is less than the predetermined threshold.

14. A phototherapy device comprising:
the control apparatus according to claim 13;
the irradiation light source configured to irradiate the affected area that is administered with a phototherapeutic agent with the therapeutic light;
wherein the processor is further configured to measure the light intensity of the affected area that is irradiated with the therapeutic light.

15. The control apparatus according to claim 6, further comprising a display configured to display a change in the measured light intensity of the affected area.

16. The phototherapy device according to claim 13, wherein
the processor is configured to determine a first period and a second period based on a change in the measured light intensity, wherein
the first period includes a state where the light intensity of the affected area significantly decreases, and the second period is a period after the first period.

17. The phototherapy device according to claim 16, wherein the processor is configured to control a display to display the determination.

18. The phototherapy device according to claim 17, wherein
the processor is configured to:
control the display to display information based on the light intensity of the affected area measured by the measurement unit in the first period,
control irradiation of the affected area with the therapeutic light and white illumination light and control the display to display an image of the affected area in the second period, and
measure the irradiation energy dose in the second period, and stop irradiation with the therapeutic light in the second period, when the irradiation energy dose reaches a predetermined dose.

19. The phototherapy according to claim 17, wherein
the processor is configured to:
control the display to display information based on the light intensity of the affected area measured by the measurement unit in the first period,
control irradiation of the affected area with the therapeutic light and white illumination light and control the display unit to display an image of the affected area in the second period, and
measure the irradiation energy dose in the second period, and issue a warning, when the irradiation energy dose reaches a predetermined amount.
